(19) United States
(12) Patent Application Publication
(10) Pub. No.: US 2006/0189698 A1
(43) Pub. Date: Aug. 24, 2006

(54) Treatment of interstitial cystitis

(76) Inventor: Jerome H. Check, Marlton, NJ (US)

Correspondence Address: DRINKER BIDDLE & REATH LLP;One Logan Square, 18th Cherry Streets, Philadelphia, PA, 19103-6996, US

(21) Appl. No.: 11/065,502

(22) Filed: Feb. 24, 2005

(51) Int. Cl. A61K 31/137

(52) U.S. Cl. 514/649

(57) ABSTRACT

The present invention relates to compositions and methods of alleviating a symptom of chronic pelvic pain syndrome with an amphetamine. Specifically, the invention includes a method of administering dextroamphetamine or a salt thereof to an animal in order to treat interstitial cystitis, vulvovaginits and/or vulvodynia. The invention also includes a composition comprising an amphetamine and at least one agent including but not limited to an antibiotic, a dietary supplement, a taste enhancer, an antiinflammatory and/or and antihistamine.

### BACKGROUND OF THE INVENTION

1. FIELD OF INVENTION

This invention relates to the treatment of chronic pelvic pain syndrome with an amphetamine. Specifically, this invention discloses the use dextroamphetamine sulfate to treat interstitial cystitis, vulvovaginitis and/or vulvodynia.

2. DESCRIPTION OF RELATED ART

One form of pain syndrome observed in the clinical setting is the chronic pelvic pain syndrome. It is a common problem and is a challenge to treat due to its unclear etiology, complex natural history, and poor response to therapy. A percentage of patients with chronic pelvic pain syndrome may have various associated problems, including bladder or bowel dysfunction, sexual dysfunction, and other systemic or constitutional symptoms. Examples of diseases included in chronic pelvic pain syndromes include interstitial cystitis, vulvovaginitis, vulvodynia and endometriosis.

Interstitial cystitis, which is of unknown etiology, is a chronic debilitating inflammatory disorder of the bladder. It is most common in women ranging in age from about thirty to sixty with onset of the condition typically occurring at about forty years of age. It is characterized by a number of urinary difficulties, such as suprapubic pressure and pain with bladder filling, urinary frequency, nocturia, dysuria, urgency and irritative voiding associated with morphological and histological changes in the bladder. The condition is characterized as “interstitial cystitis” because it is believed the condition does not affect the surface of the bladder, but instead involves the spaces between the cells, namely the interstices, in the lining of the bladder. One commonly held theory for the pathophysiology of interstitial cystitis involves changes in epithelial permeability.

Vulvovaginitis is a common disease that encompasses a variety of disorders characterized by inflammation that may be secondary to multiple causes, including infection, irritation, allergy, and systemic disease. It can affect females of all ages and its pathophysiology depends on the etiology.

Vulvodynia is characterized by unexplained vulvar pain that can cause physical disability, sexual dysfunction, limitation of normal daily activities, and psychological difficulties. The etiology of the disease is unknown; however, it has been hypothesized that viral, fungal and bacterial assaults, allergic reactions, and autoimmune responses may play a role in the disease process. Irritation of the muscles that support the uterus, bladder and rectum as well as irritation of the nerves of the vulva tissue may result in the painful symptoms associated with vulvodynia. Vulvodynia has been classified into three basic types, which include: cyclical vulvitis, where symptoms come and go; dysesthetic vulvodynia, which is characterized by constant pain, usually burning pain when touched lightly or spasmodic stabbing pains with extreme skin sensitivity; and vulvar vestibulitis, which is defined as burning, stinging, irritation of the vaginal area on a chronic basis.

Endometriosis is a disease wherein endometrium or endometrium-like tissue ectopically proliferates at a site other than the inner surface of the cavity of uterus which is the natural site thereof. The cause of endometriosis is unknown, and therapies such as separation of the adhered tissues by surgery, administration of hormones and administration of analgesics are performed. However, these therapies are symptomatic treatments, and no complete therapy exists.

Amphetamines are a derivative of phenylethylamine, which are stimulants of the central nervous system and of the sympathetic division of the peripheral nervous system. Dextroamphetamine sulfate is an amphetamine which has been used as an adjunctive treatment for narcolepsy, minimal brain dysfunction in children (e.g., hyperkinetic behavior), epilepsy and parkinsonism; however, it has not been administered to treat interstitial cystitis, vulvovaginitis or vulvodynia.

Several patents and patent applications disclose various methods for treating pain with anti-depressant drugs. U.S. Patent Application No. 2003/0203055 to Rao et al. discloses a method of treating visceral pain syndromes in a mammal. Specifically the method includes the administration of a selective norepinephrine (NE)-serotonin (5-HT) reuptake inhibitor (NSRI), for example milnacipran, to a mammal. Interstitial cystitis and vulvodynia are included among the listed visceral pain syndromes.

U.S. Pat. No. 5,441,985 to Foreman discloses a method of treating lower urinary tract disorders in mammals by utilizing compounds that inhibit the reuptake of norepinephrine. Interstitial cystitis is listed as one of the lower urinary tract disorders to be treated. This patent does not list amphetamines specifically but rather focuses on the drug tomoxetine.

U.S. Patent Application No. 2003/0199484 to Caruso discloses a method for treating urinary incontinence in a mammal by administering dextromethorphan, dextrorphan, their mixtures and/or pharmaceutically acceptable salts, alone or in combination with a pharmacologically active agent such as a sympathomimetic drug. It is also stated in the specification that dextromethorphan and dextrorphan are useful in treating interstitial cystitis.

U.S. Pat. No. 6,417,184 to Ockert discloses a triple drug therapy utilized to treat acute and chronic pain. The triple drug therapy includes at least one anxiolytic agent; at least one centrally acting alpha antiadrenergic agent; and at least one central nervous system stimulant agent. Dextroamphetamine sulfate is among the listed central nervous system stimulants.

U.S. Patent Application No. 2001/0029257 to Murdock and Williams discloses methods for transdermal administration of compositions for pain relief. Among the listed compositions for transdermal administration is dextroamphetamine.

U.S. Patent Application No. 2004/0038874 to Omoigui discloses a method for treating persistent pain, such as interstitial cystitis, by inhibiting the biochemical mediators of inflammation. Anti-depressant medications are listed as having an effect on inflammatory mediators.

Despite the foregoing developments, it is desired to provide an efficient method of treating interstitial cystitis, vulvovaginitis and vulvodynia by administration of an amphetamine, specifically dextroamphetamine sulfate. It is also desired to provide novel compositions for treating these disorders.

These and other advantages of the present invention, as well as additional inventive features, will be apparent from the description of the invention provided herein.

All references cited herein are incorporated by reference in their entireties.

### BRIEF SUMMARY OF THE INVENTION

In one aspect, the invention includes a method of alleviating at least one symptom of chronic pelvic pain syndrome, the method comprising administering an amphetamine in a therapeutically effective amount, wherein said amphetamine is not administered transdermally. In one embodiment, the amphetamine is dextroamphetamine or a salt thereof. In a preferred embodiment, the amphetamine is dextroamphetamine sulfate. In another embodiment, the symptom is selected from the group consisting of inflammation, suprapubic pressure and pain with bladder filling, urinary frequency, nocturia, dysuria, urgency, irritative voiding, pain from the urethra to the bladder and a change in epithelial permeability. In another embodiment, at least four symptoms of the pelvic pain syndrome are alleviated.

In another embodiment, the amphetamine is administered intravenously, vaginally, intracutaneously, systemically, orally or rectally. In another embodiment, the concentration of the amphetamine is from about 0.1 to about 10 mg/kg. In another embodiment, the concentration of the amphetamine is from about 0.1 to about 0.6 mg/kg. In another embodiment, the amphetamine is administered daily. In another embodiment, the amphetamine is administered twice daily. In one embodiment, the chronic pelvic pain syndrome comprises a condition selected from the group consisting of interstitial cystitis, vulvovaginitis, vulvodynia and endometriosis.

In another aspect, the invention includes a method of alleviating at least one symptom of interstitial cystitis, vulvovaginitis and/or vulvodynia in an animal, the method comprising administering a therapeutically effective amount of dextroamphetamine or a salt thereof to the animal, wherein dextromethorphan or dextrophan are not administered to the animal and the dextroamphetamine or salt thereof is administered intravenously, vaginally, intracutaneously, systemically, orally or rectally.

In another aspect, the invention includes a method of alleviating pain and urgency associated with interstitial cystitis, vulvovaginitis and/or vulvodynia in an animal, said method comprising (a) diagnosing the interstitial cystitis, vulvovaginitis and/or vulvodynia by a method selected from the group consisting of laproscopy, cystoscopy, water load test and a patient score on a patient symptom scale; and (b) administering a therapeutically effective amount of amphetamine to the animal, thereby alleviating the pain and urgency associated with interstitial cystitis, vuvlvovaginitis and/or vulvodynia In one embodiment, dextromethorphan or dextrophan are not administered to the animal. In another embodiment, the method further alleviates an increase in permeability of a bladder epithelial membrane.

In another aspect, the invention includes a composition comprising an amphetamine and at least one agent selected from the group consisting of an antibiotic, a dietary supplement, a fat supplement and an antihistamine, wherein the amphetamine is present in an amount effective to alleviate at least one symptom of chronic pelvic pain syndrome in an animal. In one embodiment, the composition further comprises an analgesic. In another embodiment, the composition further comprises an antiinflammatory. In another embodiment, the composition further comprises a taste enhancer. In another embodiment, the amphetamine is dextroamphetamine or a salt thereof. In a preferred embodiment, the amphetamine is dextroamphetamine sulfate. In another embodiment, a concentration of the dextroamphetamine sulfate is about 0.1 to about 10 mg/kg. In another embodiment, the concentration of the dextroamphetamine sulfate is from about 0.1 to about 0.6 mg/kg. In another embodiment, the composition further comprises a pharmaceutical carrier selected from the group consisting of water, finger's solutions, dextrose solution, 5% human serum albumin and liposomes. In another embodiment, the composition is in the form of a kit and further comprises instructions for use.

### DETAILED DESCRIPTION OF THE INVENTION

In one aspect, the instant invention includes a method of alleviating a symptom of chronic pelvic pain syndrome in an animal, the method comprising administering a therapeutically effective amount of an amphetamine. In one embodiment, the amphetamine is dextroamphetamine or a salt thereof. In another embodiment, the amphetamine is dextroamphetamine sulfate.

The term “chronic pelvic pain syndrome” refers to nonmenstrual pain lasting three months or longer that localizes to the pelvis and causes disability thus requiring medical or surgical treatment. Various reproductive, gastrointestinal, urologic, and neuromuscular disorders may cause or contribute to chronic pelvic pain syndrome. It is also possible to have multiple contributing factors existing in a single patient. Interstitial cystitis, vulvovaginitis, vulvodynia and/or endometriosis are diseases that have been associated with this syndrome.

Chronic pelvic pain syndrome can be diagnosed by a test selected from the group consisting of laproscopy, cystoscopy, water load test and a patient score on a patient symptom scale. These symptoms may comprise inflammation, suprapubic pressure and pain with bladder filling, urinary frequency, nocturia, dysuria, urgency, irritative voiding, pain from the urethra to the bladder and/or changes in epithelial permeability. In one embodiment, the pain from the urethra to the bladder is felt while walking or having intercourse.

Laproscopy and cystoscopy are both utilized to examine the bladder under anesthesia, in which typical findings are low bladder capacity, inflammation, Hunner's ulcer, and petecchial hemorrhages (pinpoint bleeding) of the lining of the bladder. Both the water load test and the patient symptom scale are used to examine the severity of the patient's symptoms including frequency, urgency, pain, irrative voiding, nocturia and dysuria.

The term “amphetamine” refers to any one of a group of drugs that act by stimulating the central nervous system. Like other stimulants, the short-term effects of amphetamine intake include increased heart rate, increased blood pressure, reduced appetite, dilation of the pupils, feelings of happiness and power, and reduced fatigue. Benzedrine is the trade name for the drug amphetamine and dextroamphetamine sulfate is marketed as Dexedrine or Adderall XR.

In another aspect, the invention includes novel compositions comprising an amphetamine and at least one agent selected from the group consisting of an antibiotic, a dietary supplement, a fat supplement and an antihistamine, wherein the amphetamine is present in an amount effective to alleviate at least one symptom of chronic pelvic pain syndrome in an animal. In one embodiment, the antibiotic is a sulfa drug, ampicillin, cephalathin or ciprofloxacin. In another embodiment, the composition further comprises an analgesic. In another embodiment, the composition further comprises an antiinflammatory. In a preferred embodiment, the antiinflammatory is a non-steroidal antiinflammatory drug. In another embodiment, the composition further comprises a taste enhancer. Certain embodiments exclude the addition of dextromethorphan and/or dextrophan.

The term “composition” refers to a combination of at least two compounds, for example an amphetamine and an antibiotic, wherein the compounds may be administered concurrently or sequentially with respect to one another. That is, a first compound (i.e., amphetamine) may be administered prior to, simultaneous with or subsequent to a second compound (i.e., antibiotic). The compounds coexist within the animal following administration for at least a minimum amount of time. The invention also contemplates kits comprising the compositions of the invention. The compositions of the invention may also be included in a container, pack, or dispenser together with instructions for administration.

In one embodiment, the amphetamine or composition of the invention is administered intravenously, vaginally, topically, intracutaneously, systemically, orally or rectally. Certain embodiments exclude transdermal administration. The amount of the drug, routes of delivery and timing of administration of the drug vary in accordance with the compound employed, the animal species, bodyweight, age and whether the treatment is therapeutic or prophylactic. Accordingly, in most cases dosing and dosages will be carried out according to the manufacturer's instructions or as otherwise known in the art.

Single and multiple dosing regimes are also contemplated in this invention. Multiple dosing regimes may comprise administration of two or more doses to different sites on or by different routes of administration to an animal at the same time. In one embodiment, multiple dosing regimes may comprise the administration of two or more doses of amphetamine or composition of the invention to an animal over a period of time covering hours, days and weeks. In another embodiment, the amphetamine or composition of the invention or composition of the invention is delivered daily. In another embodiment, the amphetamine or composition of the invention is delivered twice daily. In a further embodiment, the amphetamine or composition of the invention is delivered for a period of time until symptoms subside. The amount of drug in a composition may vary within a broad range, as long as effectiveness is maintained. In one embodiment, an amphetamine is administered orally to a patient at a concentration between about 0.1 to about 10 mg/kg. In another embodiment, an amphetamine is administered orally to a patient at a concentration between about 0.1 to 0.6 mg/kg. In a further embodiment, an amphetamine is administered to a patient at a concentration from about 5 to about 15 mg/kg.

In another embodiment, the amphetamine or composition of the invention is administered to an animal. In a preferred embodiment, the animal is a mammal. In a more preferred embodiment, the mammal is a human.

The compositions of the invention can be incorporated into pharmaceutical compositions suitable for administration. Such compositions typically comprise the drug(s) and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non- aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. As stated above, examples of routes of administration include parenteral, e.g., intravenous, intradermal, subcutaneous, oral (e.g., inhalation), intra-oral, transdermal (topical), vaginal, transmucosal, and rectal administration. In one embodiment, the amphetamine is administered orally. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (i.e., amphetamine) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. In one embodiment, a taste enhancer is used with the oral amphetamine composition. For the purpose of oral therapeutic administration, the active compound can be incorporated with excipients and used in the form of tablets, spansules, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, spansules, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The invention will be illustrated in more detail with reference to the following Examples, but it should be understood that the present invention is not deemed to be limited thereto.

### EXAMPLES

### Example 1

Treatment of Pelvic Pain of Bladder Origin with Dextroamphetamine Sulfate.

Two commonly used techniques to diagnose chronic pelvic pain and/or interstitial cystitis are the pelvic pain and urgency/frequency patient symptom scale and the water load test. These tests will be described briefly below.

The pelvic pain and urgency/frequency PST consists of eight questions with two of the questions each having two parts. The questions inquire about urination during the day, urination at night and the degree to which the pain bothers the women. The questions also inquire about pain with sex and urgency. The degree to which these symptoms bother the patient are ranked from 0-4 with a maximum score of 35. A total score of 10-14 equals a 74% likelihood of a positive potassium sensitivity test. A score of 21 or more equals a 91% likelihood of a positive potassium sensitivity test. A positive potassium sensitive test is more specific than cystoscopy in identifying patients with interstitial cystitis.

In a water load test, the patient drinks 1500 cc from 7-7:30 A.M. and then empties the bladder and measures her urine over the next four hours while either lying down or standing. No food, drink or smoking is allowed during this test. Patients with idiopathic edema will usually excrete 1125 cc during the standing four hour test. Typically patients excrete more during the lying down part of the test than during the standing part of the test. Healthy individuals excrete more than 1125 cc while either lying down or standing.

Two women with severe chronic bladder pain unresponsive to conventional therapy were offered a novel therapy with dextroamphetamine sulfate. In the first patient, the bladder pain started at age 35 in an intermittent manner for six months and then it became daily. The pain was described as a constant feeling of urgency and pain from the urethra to the bladder on walking. She scored an 18 on the pelvic pain and urging/frequency PST. A laparoscopy at age 30 found minimal endometriosis. A cystoscopy was negative as were repeated urine cultures. She failed to respond at all to changes in diet, in fact she claimed that ciprofloxin, ditropan and finasteride all made it worse. She failed a water load test excreting only 1000 mL urine after standing 4 hours after drinking 1500 mL.

In the second patient, the pain also began intermittently for six months starting at age 21. The pain then began occurring daily and was worse with intercourse and for three days premenstrually. The pain was described as a constant feeling of pressure in the bladder with intense pain after urination. Urine cultures were negative and empirical antibiotics provided no relief. She also failed the water load test.

Dextroamphetamine sulfate spansules (10 mg) were given twice daily to the first patient and once daily to the second patient. For the first patient, the pain completely disappeared within two days and she continued pain free for the next 1.5 years that she remained on medications. She was weaned off the drug at this time and has remained symptom free for six months. For the second patient, the pain decreased immediately and gradually improved daily for one week when it completely disappeared. She has remained pain free for four months. Interestingly, this patient forgot her pills on a short trip and the symptoms returned immediately but then disappeared immediately upon resumption of medication.

What is claimed is:

1. A method of alleviating at least one symptom of chronic pelvic pain syndrome, the method comprising administering an amphetamine in a therapeutically effective amount, wherein said amphetamine is not administered transdermally.

2. The method of claim 1, wherein the amphetamine is dextroamphetamine or a salt thereof.

3. The method of claim 1, wherein the amphetamine is dextroamphetamine sulfate.

4. The method of claim 1, wherein the symptom is selected from the group consisting of inflammation, suprapubic pressure and pain with bladder filling, urinary frequency, nocturia, dysuria, urgency, irritative voiding, pain from the urethra to the bladder and a change in epithelial permeability.

5. The method of claim 4, wherein at least four symptoms of the pelvic pain syndrome are alleviated.

6. The method of claim 1, wherein the amphetamine is administered intravenously, vaginally, intracutaneously, systemically, orally or rectally.

7. The method of claim 1, wherein the concentration of the amphetamine is from about 0.1 to about 10 mg/kg.

8. The method of claim 1, wherein the concentration of the amphetamine is from about 0.1 to about 0.6 mg/kg.

9. The method of claim 1, wherein the amphetamine is administered daily.

10. The method of claim 1, wherein the amphetamine is administered twice daily.

11. The method of claim 1, wherein the chronic pelvic pain syndrome comprises a condition selected from the group consisting of interstitial cystitis, vulvovaginitis, vulvodynia and endometriosis.

12. A method of alleviating at least one symptom of interstitial cystitis, vulvovaginitis and/or vulvodynia in an animal, the method comprising administering a therapeutically effective amount of dextroamphetamine or a salt thereof to the animal, wherein dextromethorphan or dextrophan are not administered to the animal and the dextroamphetamine or salt thereof is administered intravenously, vaginally, intracutaneously, systemically, orally or rectally.

13. A method of alleviating pain and urgency associated with interstitial cystitis, vulvovaginitis and/or vulvodynia in an animal, said method comprising:
(a) diagnosing the interstitial cystitis, vulvovaginitis and/or vulvodynia by a method selected from the group consisting of laproscopy, cystoscopy, water load test and a patient score on a patient symptom scale; and
(b) administering a therapeutically effective amount of amphetamine to the animal, thereby alleviating the pain and urgency associated with interstitial cystitis, vuvlvovaginitis and/or vulvodynia.

14. The method of claim 13, wherein dextromethorphan or dextrophan are not administered to the animal.

15. The method of claim 13, wherein the method further alleviates an increase in permeability of a bladder epithelial membrane.

16. A composition comprising an amphetamine and at least one agent selected from the group consisting of an antibiotic, a dietary supplement, a fat supplement and an antihistamine, wherein the amphetamine is present in an amount effective to alleviate at least one symptom of chronic pelvic pain syndrome in an animal.

17. The composition of claim 16 further comprising an analgesic.

18. The composition of claim 16 further comprising an antiinflammatory.

19. The composition of claim 16 further comprising a taste enhancer.

20. The composition of claim 16, wherein the amphetamine is dextroamphetamine or a salt thereof.

21. The composition of claim 16, wherein the amphetamine is dextroamphetamine sulfate.

22. The composition of claim 21, wherein a concentration of the dextroamphetamine sulfate is about 0.1 to about 10 mg/kg.

23. The composition of claim 21, wherein the concentration of the dextroamphetamine sulfate is from about 0.1 to about 0.6 mg/kg.

24. The composition of claim 16 further comprising a pharmaceutical carrier selected from the group consisting of water, finger's solutions, dextrose solution, 5% human serum albumin and liposomes.

25. The composition of claim 16, wherein the composition is in the form of a kit and further comprises instructions for use.
